(19) United States
(12) Patent Application Publication
(10) Pub. No.: US 2025/0185976 A1
(43) Pub. Date: Jun. 12, 2025

(54) METHOD AND APPARATUS FOR EXTRACTING NEUROLOGICAL DISORDER FROM EEG

(71) Applicant: Sunwoo AN, Seoul (KR)

(72) Inventor: Sunwoo AN, Seoul (KR)

(21) Appl. No.: 18/534,777

(22) Filed: Dec. 11, 2023

(51) Int. Cl. A61B 5/372; A61B 5/00; G16H 40/67; G16H 50/20

(52) U.S. Cl. CPC A61B 5/372; A61B 5/4094; A61B 5/7203; A61B 5/7267; G16H 40/67; G16H 50/20

(57) ABSTRACT

Provided is an apparatus for generating an electroencephalograph (EEG) signal, comprising a processor; and a memory comprising one or more sequences of instructions which, when executed by the processor, causes steps to be performed comprising receiving a first EEG signal and a second EEG signal; extracting a first plurality of features from the first EEG signal and a second plurality of features from the second EEG signal based on a machine learning model; generating a first reconstruction EEG signal and a second reconstruction EEG signal by swapping a same category feature among the first plurality of features and the second plurality of features based on the machine learning model so that the first EEG signal and the second EEG signal match the second reconstruction EEG signal and the first reconstruction EEG signal, respectively.

### TECHNICAL FIELD

The present disclosure relates to generating an electroencephalograph (EEG) signal using a machine learning model, more particularly, to an apparatus and method for extracting neurological disorder information in the EEG signals and detecting abnormalities such as seizure in the EEG signals using the machine learning model.

### DESCRIPTION OF THE RELATED ART

According to the World Health Organization, nearly 50 million people suffer from epilepsy worldwide, making it one of the most common neurological diseases globally (World Health Organization, 2023). Only migraine, stroke, and Alzheimer's disease are ahead in the list before epilepsy. Epilepsy is a neurological disorder characterized by abnormal brain activity, leading to recurrent seizures, and can affect individuals of any age. Each seizure manifests as sudden, uncontrolled bursts of electrical activity in the brain, resulting in a wide range of symptoms depending on the affected regions such as the stiffening of the body, loss of consciousness or breathing problems. They are caused by a sudden abnormal, self-sustained electrical discharge that occurs in the cerebral networks and usually lasts for a few minutes. The unpredictable nature of seizures poses a significant threat to the life of those who suffer from epilepsy. The potential for injury and the restrictions it imposes on daily life underscores the urgency of finding effective methods for epileptic seizure prediction.

### SUMMARY OF THE DISCLOSURE

In one aspect of the present disclosure, an apparatus for generating a biometric image, comprises a processor; and a memory comprising one or more sequences of instructions which, when executed by the processor, causes steps to be performed comprising: receiving a first EEG signal and a second EEG signal; extracting a first plurality of features from the first EEG signal and a second plurality of features from the second EEG signal based on a machine learning model; generating a first reconstruction EEG signal and a second reconstruction EEG signal by swapping a same category feature among the first plurality of features and the second plurality of features based on the machine learning model so that the first EEG signal and the second EEG signal match the second reconstruction EEG signal and the first reconstruction EEG signal, respectively.

Desirably, each of the first plurality of features and the second plurality of features may include a noise-related feature.

Desirably, the machine learning model may include a variational autoencoder having ladder networks.

Desirably, the machine learning model may be repeatedly trained to minimize a loss function for the variational autoencoder.

In another aspect of the present disclosure, an apparatus for generating a biometric image, comprises a processor; and a memory comprising one or more sequences of instructions which, when executed by the processor, causes steps to be performed comprising: receiving a first EEG signal and a second EEG signal; extracting a first plurality of features from the first EEG signal and a second plurality of features from the second EEG signal based on a machine learning model; generating a first reconstruction EEG signal and a second reconstruction EEG signal by swapping a same category feature among the first plurality of features and the second plurality of features based on the machine learning model so that the first EEG signal and the second EEG signal match the first reconstruction EEG signal and the second reconstruction EEG signal, respectively.

Desirably, each of the first plurality of features and the second plurality of features may include at least one of a seizure-related feature and a device-related feature.

Desirably, the machine learning model may include a variational autoencoder having ladder networks.

Desirably, the machine learning model may be repeatedly trained to minimize a loss function for the variational autoencoder.

Desirably, the first EEG signal may be measured by a first device, and the second EEG signal may be measured by a second device which is different the first device.

In further aspect of the present disclosure, an apparatus for anomaly detection of a biometric image comprises a processor; and a memory comprising one or more sequences of instructions which, when executed by the processor, causes steps to be performed comprising: receiving the EEG signal from a device; extracting a seizure-related feature from the EEG signal based on a first machine learning model; training a second machine learning model using the EEG signal including the seizure-related feature; and identifying probabilities of occurrence of the seizure in the EEG signal based on the pre-trained second machine learning model.

Desirably, the first machine learning model includes an encoder, and the second machine learning model includes a neural network.

Desirably, the second machine learning model uses a cross-entropy loss function to measure a difference between predicted probabilities and a true label of a given dataset.

### BRIEF DESCRIPTION OF THE DRAWINGS

References will be made to embodiments of the disclosure, examples of which may be illustrated in the accompanying figures. These figures are intended to be illustrative, and not limiting. Although the disclosure is generally described in the context of these embodiments, it should be understood that it is not intended to limit the scope of the disclosure to these particular embodiments.

FIG. 1 is an exemplary diagram for explaining a learning method to generate an electroencephalograph (EEG) signal according to embodiments of the present disclosure.

FIG. 2 is a schematic diagram of an illustrative apparatus 100 for generating an EEG signal according to embodiments of the present disclosure.

FIG. 3 is an exemplary diagram for explaining a first learning method to generate an EEG signal by an apparatus according to embodiments of the present disclosure.

FIG. 4 is an exemplary diagram for explaining a second learning method to generate an EEG signal by an apparatus according to embodiments of the present disclosure.

FIG. 5 is an exemplary diagram for explaining a third learning method to generate an EEG signal by an apparatus according to embodiments of the present disclosure.

FIG. 6 is an exemplary diagram of an apparatus for identifying a seizure in an electroencephalograph (EEG) signal according to embodiments of the present disclosure.

FIG. 7 is an exemplary flowchart showing an anomaly detection process in an electroencephalograph (EEG) signal according to embodiments of the present disclosure.

### DETAILED DESCRIPTION OF PREFERRED EMBODIMENTS

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, an apparatus, a system, a device, or a method on a tangible computer-readable medium.

Components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled,” “connected,” “linked,” or “communicatively coupled” shall be understood to include direct connections, indirect connections through one or more intermediary devices, and wireless connections.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments.

In the following description, it shall also be noted that the terms “learning” shall be understood not to intend mental action such as human educational activity of referring to performing machine learning by a processing module such as a processor, a CPU, an application processor, micro-controller, and so on.

An “attribute(s)” is defined as a group of one or more descriptive characteristics of subjects that can discriminate for a seizure. An attribute can be a numeric attribute.

The terms “comprise/include” used throughout the description and the claims and modifications thereof are not intended to exclude other technical features, additions, components, or operations.

Unless the context clearly indicates otherwise, the singular forms “a,” “an,” and “the” are intended to include the plural forms as well. Also, when description related to a known configuration or function is deemed to render the present disclosure ambiguous, the corresponding description is omitted.

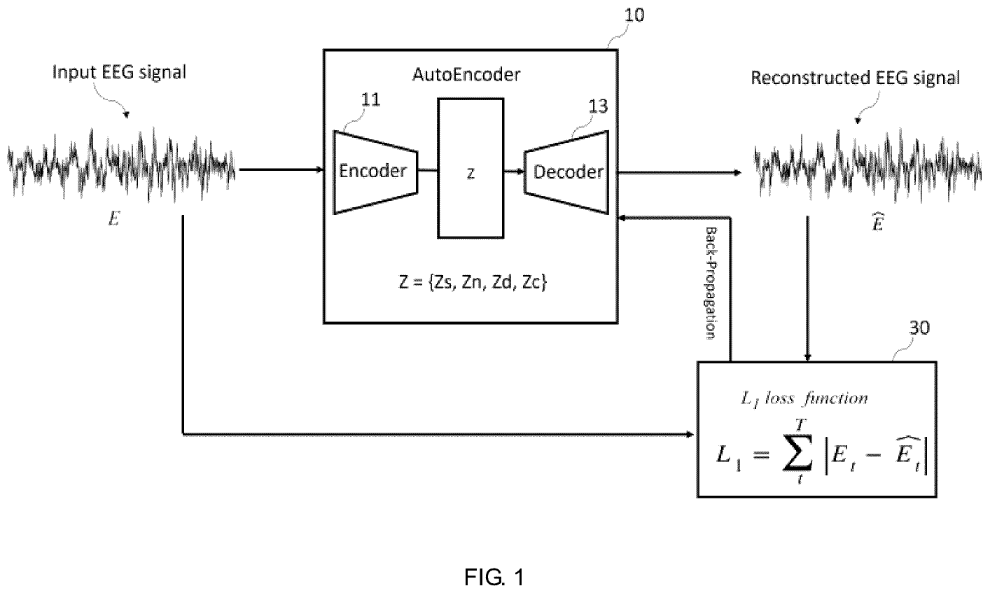

FIG. 1 is an exemplary diagram for explaining a learning method to generate an electroencephalograph (EEG) signal according to embodiments of the present disclosure.

As depicted, a machine learning model 10 may include a variational autoencoder. The variational autoencoder may include ladder networks. The autoencoder also may be a convolutional neural network (CNN) autoencoder or other types of autoencoders. The variational autoencoder may provide a probabilistic manner for describing an observation in latent space. Thus, the variational autoencoder can describe a probability distribution for each latent attribute. Each input EEG signal can be described in terms of latent attributes, such as using a probability distribution for each attribute. The variational autoencoder may use an encoder 11 and a decoder 13 during workflow operation. If high-dimensional data is input to the encoder 11 of the autoencoder, the encoder 11 performs encoding to convert the high-dimensional data into a low-dimensional latent variable Z. In embodiments, the high-dimensional data may be an EEG data, and the EEG data may include, but be not limited to, a bio-signal such as the EEG signal. In embodiments, the latent variable Z may generally be 2 to 10 dimensional data. For example, the latent variable may be a seizure-related feature Zs, a device-related feature Zd, a noise-related feature Zn, a constant feature Zc of the input EEG signal. The decoder 13 may output a reconstructed high-dimensional data by decoding the low-dimensional latent variable Z. In embodiments, the reconstructed high-dimensional data may be a reconstructed EEG signal including neurological disorder information like a seizure.

The loss calculator 30 may calculate a difference between a comparison data stored in a memory (not shown) and the reconstructed high-dimensional data using a loss function L1. The loss function L1 may be calculated as follows.

$${L}_{1} = {\sum }_{t}^{T} \vert {E}_{t} - \vert$$

Here, T denotes the total time of the dataset, and Eₜ and Êₜ denotes a point in the original data and reconstructed data, respectively. For each second of the EEG signals, the difference between the original data and reconstructed data is calculated and summed.

The autoencoder 10 may be repeatedly trained to minimize the loss function using a backpropagation algorithm. In embodiments, the comparison data may be the input EEG signal. The input EEG signal may be a signal capable of detecting neurological disorder information from a single modality signal. In embodiments, the loss function may use a mean squared error. In this case, the mean squared error may be the sum of squared differences between the epileptiform discharges (ED) of the reconstructed EEG signal and the epileptiform discharges (ED) of the input EEG signal. The epileptiform discharges may be “spikes” or “sharp waves,” which can be indicators of some abnormality or problem within the subject.

Thus, the learning method for generating an electroencephalograph signal using the unsupervised machine learning algorithm of conditional variational autoencoder may be performed by a computing device 110 described below.

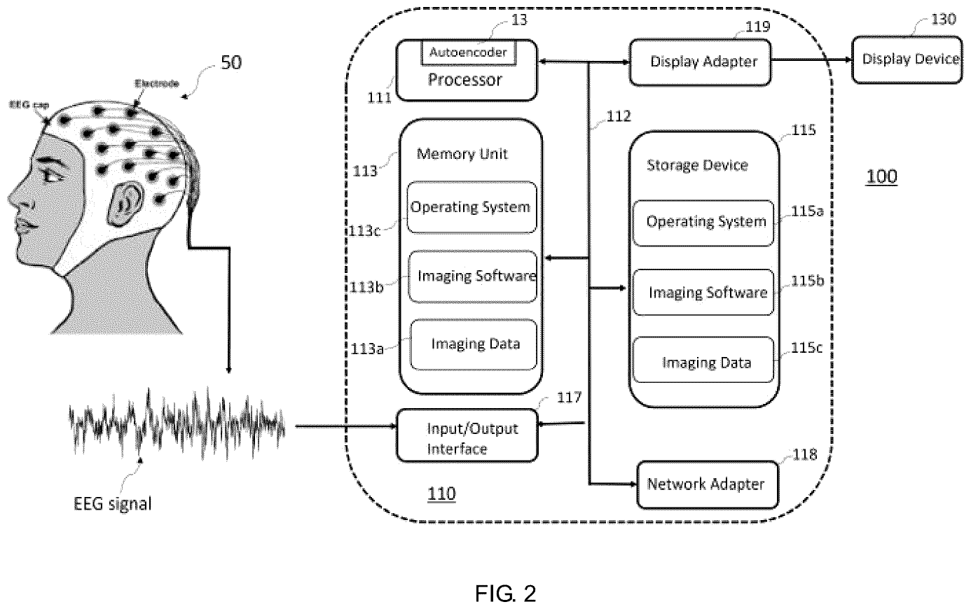

FIG. 2 is a schematic diagram of an illustrative apparatus 100 for generating an EEG signal according to embodiments of the present disclosure.

As depicted, the apparatus 100 may include an EEG device 50, a computing device 110, a display device 130. In embodiments, the computing device 110 may include, but is not limited thereto, one or more processor 111, a memory unit 113, a storage device 115, an input/output interface 117, a network adapter 118, a display adapter 119, and a system bus 112 connecting various system components to the memory unit 113. In embodiments, the apparatus 100 may further include communication mechanisms as well as the system bus 112 for transferring information. In embodiments, the communication mechanisms or the system bus 112 may interconnect the processor 111, a computer-readable medium, a short range communication module (e.g., a Bluetooth, a NFC), the network adapter 118 including a network interface or mobile communication module, the display device 130 (e.g., a CRT, a LCD, etc.), an input device (e.g., a keyboard, a keypad, a virtual keyboard, a mouse, a trackball, a stylus, a touch sensing means, etc.) and/or subsystems. In embodiments, the EEG device 50 may include an EEG cap and an electrode installed on the EEG cap. The EEG signal acquired by the EEG device 50 may include seizure-related features such as frequency, amplitude, noise of the signal. The EEG signal may be stored in the memory unit 113 or the storage device 115 in time series or may be provided to the processor 111 through the input/output interface 117 and processed based on a machine learning model 13.

In embodiments, the processor 111 is, but is not limited to, a processing module, a Computer Processing Unit (CPU), an Application Processor (AP), a microcontroller, and/or a digital signal processor. In addition, the processor 111 may communicate with a hardware controller such as the display adapter 119 to display a user interface on the display device 130. In embodiments, the processor 111 may access the memory unit 113 and execute commands stored in the memory unit 113 or one or more sequences of instructions to control the operation of the apparatus 100. The commands or sequences of instructions may be read in the memory unit 113 from computer-readable medium or media such as a static storage or a disk drive, but is not limited thereto. In alternative embodiments, a hard-wired circuitry which is equipped with a hardware in combination with software commands may be used. The hard-wired circuitry can replace the soft commands. The instructions may be an arbitrary medium for providing the commands to the processor 111 and may be loaded into the memory unit 113.

In embodiments, the system bus 112 may represent one or more of several possible types of bus structures, including a memory bus or memory controller, a peripheral bus, an accelerated graphics port, and a processor or local bus using any of a variety of bus architectures. For instance, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. In embodiments, the system bus 112, and all buses specified in this description can also be implemented over a wired or wireless network connection.

A transmission media including wires of the system bus 112 may include at least one of coaxial cables, copper wires, and optical fibers. For instance, the transmission media may take a form of sound waves or light waves generated during radio wave communication or infrared data communication.

In embodiments, the apparatus 100 may transmit or receive the commands including messages, data, and one or more programs, i.e., a program code, through a network link or the network adapter 118. In embodiments, the network adapter 118 may include a separate or integrated antenna for enabling transmission and reception through the network link. The network adapter 118 may access a network and communicate with a remote computing device.

In embodiments, the network may be, but is not limited to, more than one of LAN, WLAN, PSTN, and cellular phone networks. The network adapter 118 may include at least one of a network interface and a mobile communication module for accessing the network. In embodiments, the mobile communication module may be accessed to a mobile communication network for each generation such as 2G to 5G mobile communication network.

In embodiments, on receiving a program code, the program code may be executed by the processor 111 and may be stored in a disk drive of the memory unit 113 or in a non-volatile memory of a different type from the disk drive for executing the program code.

In embodiments, the computing device 110 may include a variety of computer-readable medium or media. The computer-readable medium or media may be any available medium or media that is accessible by the computing device 110. For example, the computer-readable medium or media may include, but is not limited to, both volatile and non-volatile media, removable or non-removable media.

In embodiments, the memory unit 113 may store a driver, an application program, data, and a database for operating the apparatus 100 therein. In addition, the memory unit 113 may include a computer-readable medium in a form of a volatile memory such as a random-access memory (RAM), a non-volatile memory such as a read only memory (ROM), and a flash memory. For instance, it may be, but is not limited to, a hard disk drive, a solid-state drive, and/or an optical disk drive.

In embodiments, each of the memory unit 113 and the storage device 115 may be program modules such as the imaging software 113b, 115b and the operating systems 113c, 115c that can be immediately accessed so that a data such as the imaging data 113a, 115a is operated by the processor 111.

In embodiments, the machine learning model 13 may be installed into at least one of the processor 111, the memory unit 113 and the storage device 115. In embodiments, the processor 111 may generate a reconstructed EEG signal from the input EEG signal stored in the memory unit 113 or the storage device 115 or provided from the EEG device 50 based on the trained machine learning model 13. In this case, the processor 111 may reconstruct the EEG signal using the machine learning model 13 trained by the learning method in described conjunction with FIG. 1. The reconstructed EEG signal generated by the processor 111 has substantially the same information as the information of the input EEG signal described in FIG. 1. The machine learning model 13 may be trained using unsupervised machine learning described in FIG. 1. The machine learning model 13 may include a variational autoencoder that includes ladder networks.

Thus, the apparatus 100 may generate the reconstructed EEG signal from the input EEG signal based on the learned model to detect neurological disorder information from the reconstructed EEG signal in real time in the medical environment.

If the apparatus 100 includes more than one computing device 110, then the different computing devices may be coupled to each other such that images, data, information, instructions, etc. can be sent between the computing devices. For example, one computing device may be coupled to additional computing device(s) by any suitable transmission media, which may include any suitable wired and/or wireless transmission media known in the art. Computing devices that implement at least one or more of the methods, functions, and/or operations described herein may comprise an application or applications operating on at least one computing device. The computing device may comprise one or more computers and one or more databases. The computing device may be a single device, a distributed device, a cloud-based computer, or a combination thereof.

It shall be noted that the present disclosure may be implemented in any instruction-execution/computing device or system capable of processing data, including, without limitation laptop computers, desktop computers, and servers. The present invention may also be implemented into other computing devices and systems. Furthermore, aspects of the present invention may be implemented in a wide variety of ways including software (including firmware), hardware, or combinations thereof. For example, the functions to practice various aspects of the present invention may be performed by components that are implemented in a wide variety of ways including discrete logic components, one or more application specific integrated circuits (ASICs), and/or program-controlled processors. It shall be noted that the manner in which these items are implemented is not critical to the present invention.

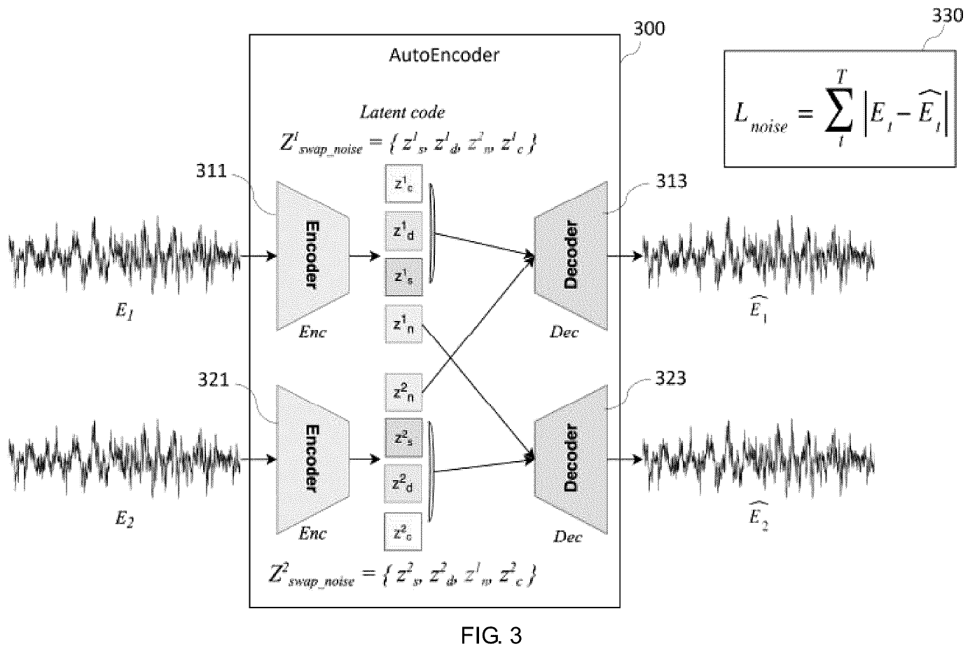

FIG. 3 is an exemplary diagram for explaining a first learning method to generate an EEG signal by an apparatus according to embodiments of the present disclosure.

As depicted, the autoencoder 300 installed in the present processor may include a first encoder 311, a second encoder 321, a first decoder 313 and a second decoder 323. A first EEG signal E₁ and a second EEG signal E₂ measured with the EEG device may input into the first encoder 311 and the second encoder 321, respectively. In embodiments, the first EEG signal and the second EEG signal measured with the same EEG device may be same signal but may be formed to suppress a noise contained in either one by a wavelet-inverse wavelet transformation. Each of the first encoder 311 and the second encoder 321 may extract a first plurality of features from the first EEG signal and a second plurality of features from the second EEG signal. Each of the first and second plurality of features may be a seizure-related feature z¹ₛ, z²ₛ, a device-related feature z¹_{d}, z²_{d}, a noise-related feature z¹ₙ, z²ₙ, and a common feature z¹_{c}, z²_{c}. The first and second plurality of features may be mapped into the shared latent space Z corresponding to a class-dependent transformation. The features of the EEG signal may be mapped into the latent space by swapping a same category feature such as the noise-related feature z¹ₙ, z²ₙ. The first decoder 313 may generate a first reconstruction EEG signal Ê₁ including the swapped noise feature z²ₙ and the second decoder 323 may also generate a second reconstruction EEG signal Ê₂ including the swapped noise feature z¹ₙ. In this case, a loss calculator 330 may calculate a difference between a first EEG signal E₁ and the second reconstruction EEG signal Ê₂, and may also calculate a difference between a second EEG signal E₂ and the first reconstruction EEG signal Ê₁ using a loss function L noise. The loss function Lₙₒᵢₛₑ may be calculated as follows.

$${L}_{noise} = {{\sum }^{T}}_{i} \vert {E}_{t} - \vert$$

Here, T denotes the number of samples in the inputted EEG signal, and Eₜ and Êₜ denotes the inputted EEG signal and its reconstructed EEG signals based on the swapped features, respectively.

The autoencoder 300 may be repeatedly trained to minimize the loss function using a backpropagation algorithm whereby the first EEG signal E₁ and the second EEG signal E₂ may identically match the second reconstruction EEG signal Ê₂ and the first reconstruction EEG signal Ê₁, respectively.

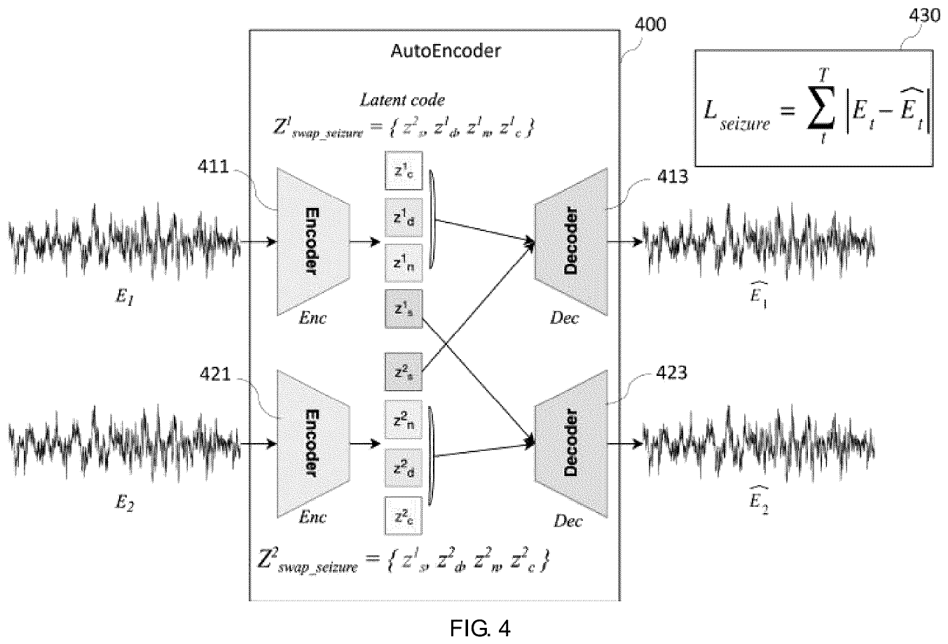

FIG. 4 is an exemplary diagram for explaining a second learning method to generate an EEG signal by an apparatus according to embodiments of the present disclosure.

As depicted, the autoencoder 400 installed in the present processor may include a first encoder 411, a second encoder 421, a first decoder 413 and a second decoder 423. A first EEG signal E₁ and a second EEG signal E₂ may input into the first encoder 411 and the second encoder 421, respectively. In embodiments, each of the first EEG signal E₁ and a second EEG signal E₂ may be measured with the different EEG devices, respectively. Each of the first encoder 411 and the second encoder 421 may extract a first plurality of features from the first EEG signal and a second plurality of features from the second EEG signal. Each of the first and second plurality of features may be a seizure-related feature z¹ₛ, z²ₛ, a device-related feature z¹_{d}, z²_{d}, a noise-related feature z¹ₙ, z²ₙ, and a common feature z¹_{c}, z²_{c}. The first and second plurality of features may be mapped into the shared latent space Z corresponding to a class-dependent transformation. At this time, the features of the EEG signal may be mapped into the latent space by swapping a same category feature such as the seizure-related feature z¹ₛ, z²ₛ. The first decoder 413 may generate a first reconstruction EEG signal Ê₁ including the swapped seizure feature z²ₛ and the second decoder 423 may also generate a second reconstruction EEG signal Ê₂ including the swapped seizure feature z¹ₛ. In this case, a loss calculator 430 may calculate a difference between a first EEG signal E₁ and the first reconstruction EEG signal Ê₁, and may also calculate a difference between a second EEG signal E₂ and the second reconstruction EEG signal Ê₂ using a loss function L seizure. The loss function L_{seizure} may be calculated as follows.

$${L}_{seizure} = {{\sum }^{T}}_{i} \vert {E}_{t} - \vert$$

Here, T denotes the number of samples in the inputted EEG signal, and Eₜ and Êₜ denotes the inputted EEG signal and its reconstructed EEG signals based on the swapped features, respectively.

The autoencoder 400 may be repeatedly trained to minimize the loss function using a backpropagation algorithm whereby the first EEG signal E₁ and the second EEG signal E₂ may identically match the first reconstruction EEG signal Ê₁ and the second reconstruction EEG signal Ê₂, respectively.

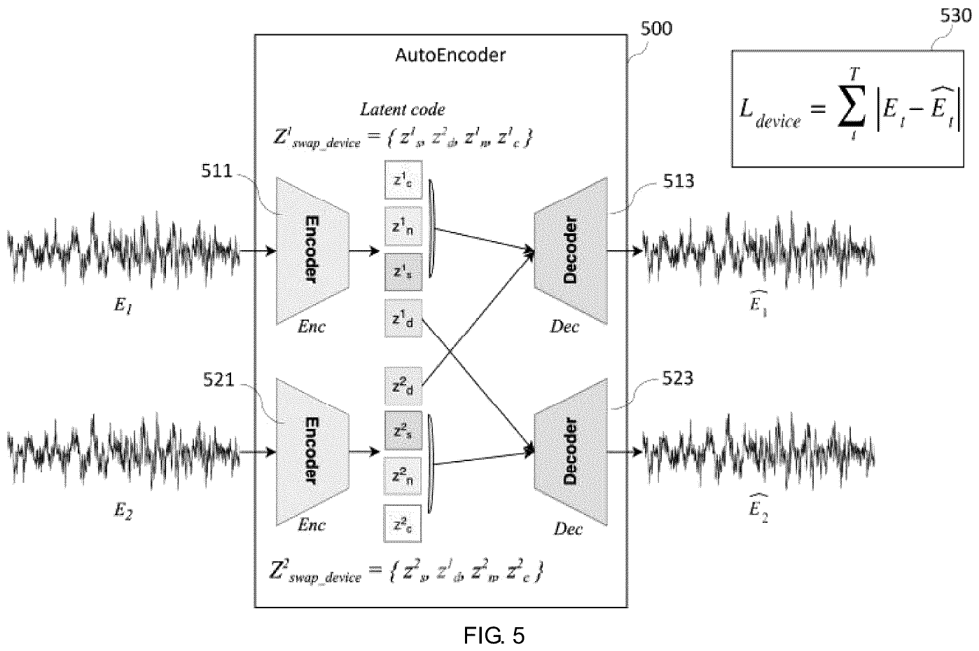

FIG. 5 is an exemplary diagram for explaining a third learning method to generate an EEG signal by an apparatus according to embodiments of the present disclosure.

As depicted, the autoencoder 500 installed in the present processor may include a first encoder 511, a second encoder 521, a first decoder 513 and a second decoder 523. A first EEG signal E₁ and a second EEG signal E₂ may input into the first encoder 511 and the second encoder 521, respectively. In embodiments, each of the first EEG signal E₁ and a second EEG signal E₂ may be measured with the different EEG devices, respectively. Each of the first encoder 511 and the second encoder 521 may extract a first plurality of features from the first EEG signal and a second plurality of features from the second EEG signal. Each of the first and second plurality of features may be a seizure-related feature z¹ₛ, z²ₛ, a device-related feature z¹_{d}, z²_{d}, a noise-related feature z¹ₙ, z²ₙ, and a common feature z¹_{c}, z²_{c}. The first and second plurality of features may be mapped into the shared latent space Z corresponding to a class-dependent transformation. At this time, the features of the EEG signal may be mapped into the latent space by swapping a same category feature such as the device-related feature z¹_{d}, z²_{d}. The first decoder 513 may generate a first reconstruction EEG signal Ê₁ including the swapped seizure feature z²ₛ and the second decoder 523 may also generate a second reconstruction EEG signal Ê₂ including the swapped seizure feature z¹ₛ. In this case, a loss calculator 530 may calculate a difference between a first EEG signal E₁ and the first reconstruction EEG signal Ê₁, and may also calculate a difference between a second EEG signal E₂ and the second reconstruction EEG signal Ê₂ using a loss function L device. The loss function L_{device} may be calculated as follows.

$${L}_{device} = {{\sum }^{T}}_{i} \vert {E}_{t} - \vert$$

Here, T denotes the number of samples in the inputted EEG signal, and Eₜ and Êₜ denotes the inputted EEG signal and its reconstructed EEG signals based on the swapped features, respectively.

The autoencoder 500 may be repeatedly trained to minimize the loss function using a backpropagation algorithm whereby the first EEG signal E₁ and the second EEG signal E₂ may identically match the first reconstruction EEG signal Ê₁ and the second reconstruction EEG signal Ê₂, respectively.

Thus, the apparatus according to the present disclosure may generate the first reconstruction EEG signal Ê₁ and the second reconstruction EEG signal Ê₂ by minimizing each of the above-described loss functions, but may also generate the first reconstruction EEG signal Ê₁ and the second reconstruction EEG signal Ê₂ by using all loss functions as follows.

$$L all = {L}_{seizure} + α * {L}_{device} + β * {L}_{noise}$$

Here, α and β are variables that assign weights to each loss function. The variables may be assigned for optimal values.

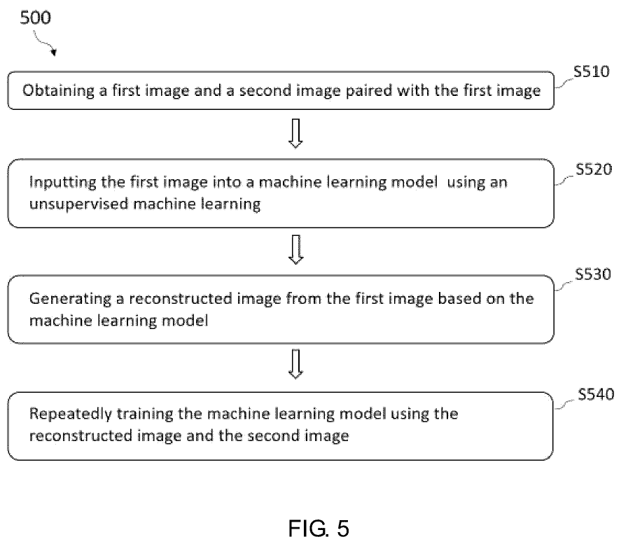

FIG. 6 is an exemplary diagram of an apparatus for identifying a seizure in an electroencephalograph (EEG) signal according to embodiments of the present disclosure.

As depicted, the apparatus 100 may include a processor 600 for running a plurality of machine learning models. The plurality of machine learning models may include a first machine learning model 610 for extracting a plurality of features from an input EEG signal and a second machine learning 630 for identifying probabilities of occurrence of the seizure in the input EEG signal. Specifically, the plurality of the features may be extracted through an encoder of the first machine learning 610 and include various features such as a seizure-related feature Zs, a device-related feature Zd, and a noise-related feature Zn in the input EEG signal as described above. Among the various features, the seizure-related feature may be taken as an input date and processed by the second machine learning model 630. The second machine learning model 630 may output the probabilities of occurrence of the seizure in the EEG signal as a neural network like a seizure classifier SCL. In this case, the processor 600 may use a cross-entropy loss function to improve the performance of the seizure classifier. The cross-entropy loss function may measure the difference between predicted probabilities of seizure and the true labels of a given dataset. The cross-entropy loss function L_{CE} may be calculated as follows.

$${L}_{CE} = - {\sum }_{i = 1}^{output size} {y}_{i} \times log$$

Here, yi and [symbol] denotes the true output and predicted output, respectively. The log function may be utilized because log values increase rapidly as the predicted probability approaches 0, the wrong prediction. This function can increase the accuracy as the loss will be much greater as the prediction reaches a completely wrong value.

Meanwhile, before the cross-entropy loss function is computed, an activation function such as softmax function may be applied to the raw scores. As such, the raw scores are normalized into probabilities between 0 and 1. For example, the softmax function is as follows.

$$S ( {x}_{i} ) = \frac{{e}^{{x}_{i}}}{{\sum }_{j = 1}^{n} {e}^{{x}_{i}}}$$

Here, xᵢ denotes the ith raw score. The equation applies exponential functions, an increasing function only including positive numbers to change the scores into positive numbers without changing the strict inequality of them since probabilities cannot be negative. Then, the scores are normalized into probabilities that sum to 1 by dividing each score by the sum of all the scores.

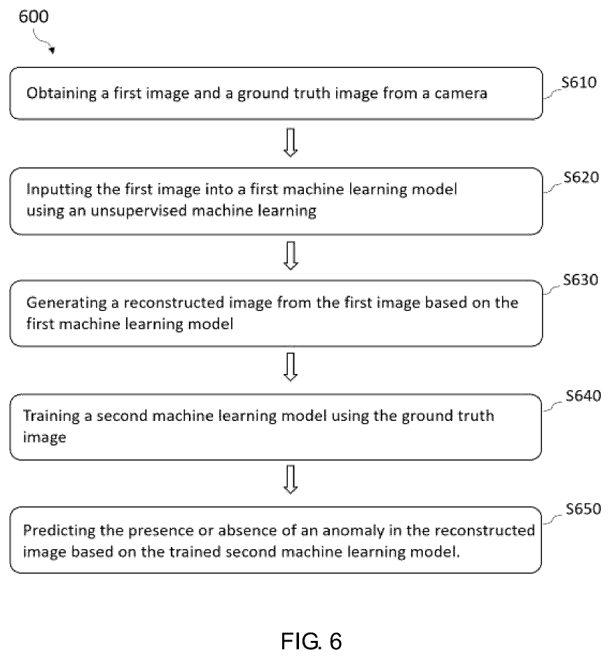

FIG. 7 is an exemplary flowchart showing an anomaly detection process in an electroencephalograph (EEG) signal according to embodiments of the present disclosure. The anomaly detection process 700 may be performed by any suitable computing device(s). At step S710, an EEG signal is obtained from an EEG device of an apparatus to make a training set. In embodiments, the EEG signal may be multiple signals obtained from a same EEG device or different EEG devices. If the multiple signals are measured by the same EEG device, one of the multiple signals may be formed to suppress a noise of the signal by a wavelet-inverse wavelet transformation. At step S720, if the EEG signal is input into a first machine learning model, the first machine learning model using unsupervised machine learning is trained using the EEG signal. The first machine learning model may include a variational autoencoder that includes an encoder and a decoder. At step S730, the variational autoencoder may extract seizure-related features such as a frequency, an amplitude, a noise in the EEG signal, and generate a reconstructed EEG signal from the EEG signal. The variational autoencoder may include ladder networks. At step S740, a second machine learning model may be trained using the EEG signal with the seizure-related features. The second machine learning model may use unsupervised machine learning or supervised machine learning to classify a seizure. At step S750, if the EEG signal is input into the second machine learning model, the trained second learning model may predict whether there is the presence or absence of an anomaly in the EEG signal. In this case, the trained second learning model may use a cross-entropy loss function to measure a difference between predicted probabilities and a true label of a given dataset.

Embodiments of the present disclosure may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store, or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present disclosure. One skilled in the art will also recognize that a number of the elements described above may be physically and/or functionally separated into sub-modules or combined together.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. An apparatus for generating an electroencephalograph (EEG) signal, comprising:
a processor; and
a memory comprising one or more sequences of instructions which, when executed by the processor, causes steps to be performed comprising:
receiving a first EEG signal and a second EEG signal;
extracting a first plurality of features from the first EEG signal and a second plurality of features from the second EEG signal based on a machine learning model;
generating a first reconstruction EEG signal and a second reconstruction EEG signal by swapping a same category feature among the first plurality of features and the second plurality of features based on the machine learning model so that the first EEG signal and the second EEG signal match the second reconstruction EEG signal and the first reconstruction EEG signal, respectively.

2. The apparatus of claim 1, wherein each of the first plurality of features and the second plurality of features includes a noise-related feature.

3. The apparatus of claim 1, wherein the machine learning model includes a variational autoencoder having ladder networks.

4. The apparatus of claim 3, wherein the machine learning model is repeatedly trained to minimize a loss function for the variational autoencoder.

5. An apparatus for generating an electroencephalograph (EEG) signal, comprising:
a processor; and
a memory comprising one or more sequences of instructions which, when executed by the processor, causes steps to be performed comprising:
receiving a first EEG signal and a second EEG signal;
extracting a first plurality of features from the first EEG signal and a second plurality of features from the second EEG signal based on a machine learning model;
generating a first reconstruction EEG signal and a second reconstruction EEG signal by swapping a same category feature among the first plurality of features and the second plurality of features based on the machine learning model so that the first EEG signal and the second EEG signal match the first reconstruction EEG signal and the second reconstruction EEG signal, respectively.

6. The apparatus of claim 5, wherein each of the first plurality of features and the second plurality of features includes at least one of a seizure-related feature and a device-related feature.

7. The apparatus of claim 5, wherein the machine learning model includes a variational autoencoder having ladder networks.

8. The apparatus of claim 7, wherein the machine learning model is repeatedly trained to minimize a loss function for the variational autoencoder.

9. The apparatus of claim 5, wherein the first EEG signal is measured by a first device, and the second EEG signal is measured by a second device which is different the first device.

10. An apparatus for identifying a seizure in an electroencephalograph (EEG) signal, comprising:
a processor; and
a memory comprising one or more sequences of instructions which, when executed by the processor, causes steps to be performed comprising:
receiving the EEG signal from a device;
extracting a seizure-related feature from the EEG signal based on a first machine learning model;
training a second machine learning model using the EEG signal including the seizure-related feature; and
identifying probabilities of occurrence of the seizure in the EEG signal based on the pre-trained second machine learning model.

11. The apparatus of claim 10, wherein the first machine learning model includes an encoder, and the second machine learning model includes a neural network.

12. The apparatus of claim 10, wherein the second machine learning model uses a cross-entropy loss function to measure a difference between predicted probabilities and a true label of a given dataset.
